(19) United States
(12) Patent Application Publication
(10) Pub. No.: US 2024/0424281 A1
(43) Pub. Date: Dec. 26, 2024

(54) ELASTOMERIC VEIN GRAFT PREPARATION PUMP SYSTEM AND METHOD OF USE

(71) Applicant: Quest Medical, Inc., Allen, TX (US)

(72) Inventors: Steve Parnis, Missouri City, TX (US); Andrew Nelson, Dallas, TX (US); Jeffrey Albertsen, McKinney, TX (US)

(21) Appl. No.: 18/749,061

(22) Filed: Jun. 20, 2024

Related U.S. Application Data
(60) Provisional application No. 63/522,577, filed on Jun. 22, 2023.

(51) Int. Cl. A61M 60/148; A61M 60/268

(52) U.S. Cl. CPC A61M 60/148; A61M 60/268

(57) ABSTRACT

An elastomeric vein graft preparation pump. The elastomeric vein graft preparation pump can include an elastomeric compliant body configured to contain a fluid, a fluid conduit coupled to the elastomeric compliant body, and a control valve disposed through the fluid conduit. The control valve is configured to regulate a flow of the fluid through the fluid conduit. The elastomeric compliant body provides an elastic recoil pressure based on a volume of the fluid contained in the elastomeric compliant body, and the fluid flows through the fluid conduit at a pressure based on the elastic recoil pressure.

### CROSS-REFERENCE TO RELATED APPLICATIONS

This application claims the benefit of U.S. Application Ser. No. 63/522,577 filed on Jun. 22, 2023, the entirety of which is incorporated by reference.

### BACKGROUND

### Technical Field

The present disclosure is generally related to the field of vein grafting and more particularly to a novel elastomeric vein graft preparation pump for preparing veins for grafting, as well as methods of use and methods of manufacture thereof.

### Description of Related Art

Coronary artery disease (CAD) is caused by the buildup of plaque in the arteries of the heart, which results in stenotic lesions (i.e., narrow or constricted areas) in cardiac arteries that can impede normal blood flow. There are two methods by which stenotic lesions of the cardiac arteries are revascularized: percutaneous coronary intervention (PCI) and coronary artery bypass surgery, also called coronary artery bypass grafting (CABG). PCI is a non-surgical procedure used to treat narrowing of the coronary arteries using a combination of coronary angioplasty and stenting. Conversely, a CABG procedure is a surgical procedure using arteries or veins harvested from other parts of the body to bypass narrowing areas of the coronary arteries. After performing such a bypass, CABG restores adequate blood supply to the heart, thereby slowing the progression of CAD and increasing life expectancy for the patient.

A common CABG procedure involves a saphenous vein graft (SVG) for the bypassing of narrowed areas of the arteries. The great saphenous vein (or long saphenous vein) is a large, subcutaneous, superficial vein of the leg. It is the longest vein in the body, running along the length of the lower limb for returning blood from the foot, leg, and thigh to the deep femoral vein. Unfortunately, long-term patency (i.e., remaining sufficiently open) of SVGs is a common problem and vein graft failure (VGF) is reportedly as high as 45% at 18 months after surgery. It is believed that the high long-term failure rate is due to injury of the SVG during preparation prior to grafting, which may promote VGF.

Several components of the vein harvesting and engraftment process result in damage to the tunica intima (i.e., the innermost layer) of vein grafts. For example, veins are damaged during harvesting and engraftment due to ischemia (restriction in blood flow/supply) and reperfusion (return of blood flow/supply following a period of lack of oxygen). During and after harvesting, veins go through a period of ischemia. After engraftment, veins go through reperfusion. Both ischemia and reperfusion damage endothelial cells (cells that line the interior surface of the vein) and smooth muscle cells (the cells present in the layer surrounding the tunica intima).

Veins are also damaged during engraftment due to exposure to an environment to which they are not naturally adapted. Veins are naturally adapted to an environment of low pressure and low blood flow. During vein grafting, veins are exposed to high pressure and flow as they are transferred and integrated into the arterial circulation. This exposure to high pressure and flow causes increases shear stress and wall tension, which further damages the endothelial layer and smooth muscle cells. Over time, such continued damage results in luminal loss that makes the graft more susceptible to atherosclerosis. Progressive atherosclerosis is the primary cause of late vein graft failure.

Some structural changes of the tunica intima are necessary during preparation of the graft to prevent thickening (i.e., intimal hyperplasia) and vein wall remodeling after engraftment. Procedures to accomplish these structural changes typically involve distension (i.e., stretching) of the veins using fluid pressure. When elevated or unregulated pressure is used, these preparation procedures can also contribute to vein graft damage and VGF.

Conventional preparation of saphenous veins for an SVG typically involves the manual distension of the veins using fluid pressure. For example, U.S. Pat. No. 3,958,557 provides a device and method for preparing a blood vessel, such as the saphenous vein, for use as a coronary bypass graft. FIG. 1 illustrates an isometric view of a cannula 10 constructed in accordance with the principles disclosed in the U.S. Pat. No. 3,958,557. This conventional cannula 10 is comprised of a medical grade polyethylene having the flexibility and softness required to prevent trauma to the intima of the vein in which it is used. The cannula 10 has a hub 14 at the proximal end thereof which is cylindrical and terminates in a planar surface 16. The hub 14 is connected to a body 12 that is diametrically enlarged to form a shoulder 18 in a plane parallel to the surface 16. The hub 14 is sized to accommodate coupling with surgical tubing (not shown), which is press-fit on the hub 14 and abuts shoulder 18. The body 12 is frustoconical in configuration, the forwardly tapering ramp surface 20 merging with stylet 22, which forms the distal end of the cannula 10. The stylet 22 has a very gradual taper from the body 12 to the distal end 24. The external dimension of the stylet 22 is selected to be essentially the normal internal diameter of a suitable vein segment for use in coronary bypass. The stylet 22 includes a peripheral flange 26 spaced slightly behind the distal end 24. The peripheral flange 26 has a forwardly tapering ramp surface 28 that facilitates insertion of the stylet 22 into a suitable vein segment 30. The flange 26 terminates in a shoulder 32 which permits pressure-tight ligation, as described below.

FIG. 2 illustrates a side, partial cross-section view of the conventional cannula 10 taught in U.S. Pat. No. 3,958,557. The stylet 22 has an annular bore 34 that opens at the distal end 24 and is also in open communication with hollow 38 in the hub 14. The hollow 38 is coaxial with the bore 34 and is provided with a Luer taper so as to form a female coupling. Thus, the cannula 10 is press-coupled to an irrigation instrument such as a syringe 40. The selected portion of the vein 30 is then severed and the stylet 22 inserted within the lumen of the resected vein 30 until the vein 30 is pressed tightly upon the ramp surface 20. Thereafter, temporary ligation is effected by tying suture 42 tightly around the vein 30 near the flange 26. Ligation will have the effect of creating a fluid seal between the vein segment 30 and the cannula 10.

Thereafter, a syringe 40 is used for irrigating purposes to determine whether occlusions or clots remain in the vein segment 30 and, if so, to accommodate flushing of the vein 30. A distant portion of the vein segment 30 can be clamped and manual hydrostatic pressure communicated to the vein through the cannula 10 with the syringe 40. This applied pressure is used to detect leaks in the vein segment 30, particularly at the severed tributary sites. This pressure also serves to determine the distensibility index of the vein 30. If the vein 30 distends too easily under pressure, it is not suitable for the coronary artery bypass. Assuming the vein 30 has been proven under this manual pressurization, the vein 30 is used for the coronary artery bypass.

However, data has shown that acute damage to the vein graft intima can result from extensive distension of such veins during repair of the severed tributaries of the vein graft, but that this does not necessarily occur at pressure equivalent to normal arterial pressure. However, manual pressure distension does not limit or otherwise regulate the pressure of fluid applied and does not circulate the fluid during preparation. Consequently, intimal damage, although unknown to the clinician at the time, is often caused to the grafted vein when prepared using such manual procedures due to high pressurization as a result of unregulated manual pressurization techniques found in U.S. Pat. No. 3,958,557 as well as other conventional approaches. Accordingly, what is needed in the art is a device and related method for limiting the pressure of the vein graft during preparation for a vein graft to be used in a coronary artery bypass procedure that does not suffer from the deficiencies found with conventional vein preparation approaches. The disclosed principles provide such a unique device, methods of use, and methods of manufacture thereof.

### BRIEF SUMMARY

This summary provides a discussion of aspects of certain embodiments of the invention. It is not intended to limit the claimed invention or any of the terms in the claims. The summary provides some aspects but there are aspects and embodiments of the invention that are not discussed here.

In one aspect, an elastomeric vein graft preparation pump is provided. The elastomeric vein graft preparation pump can include an elastomeric compliant body configured to contain a fluid, a fluid conduit coupled to the elastomeric compliant body, and a control valve disposed through the fluid conduit. The control valve is configured to regulate a flow of the fluid through the fluid conduit. The elastomeric compliant body provides an elastic recoil pressure based on a volume of the fluid contained in the elastomeric compliant body, and the fluid flows through the fluid conduit at a pressure based on the elastic recoil pressure.

In another aspect, a method of operating an elastomeric vein graft preparation pump is provided. The method includes at least partially filling an elastomeric compliant body with a fluid, coupling the elastomeric compliant body to a first end of a housing that defines a fluid conduit extending through the housing from the first end to a second end, coupling a vessel cannula to the second end of the housing, and coupling a first end of a vessel to the vessel cannula, wherein the vessel is sealed at a second end. The housing includes a control valve disposed through the fluid conduit, and the control valve is configured to selectively release the fluid from the elastomeric compliant body.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawing, in which:

FIG. 1 illustrates an isometric view of a cannula constructed in accordance with the prior art, and used in the irrigation of a vein harvested for a grafting procedure;

FIG. 2 illustrates a side, partial cross-section view of the conventional cannula illustrated in FIG. 1;

FIG. 3 illustrates a perspective view of an exemplary embodiment of an elastomeric vein graft preparation pump designed and constructed in accordance with the disclosed principles;

FIG. 4 illustrates a perspective view of an exemplary embodiment of an elastomeric compliant body for use with the elastomeric vein graft preparation pump according to the present disclosure;

FIG. 5 illustrates a perspective view of an exemplary embodiment of an overfill prevention device for use with the elastomeric vein graft preparation pump according to the present disclosure;

FIGS. 6A and 6B illustrate perspective views of one embodiment of a control valve for use with the vein graft preparation pump according to the present disclosure;

FIG. 7 illustrates a perspective view of the elastomeric vein graft preparation pump in a loaded, unused state; and

FIG. 8 illustrates a perspective view of the elastomeric vein graft preparation in a deployed, used state.

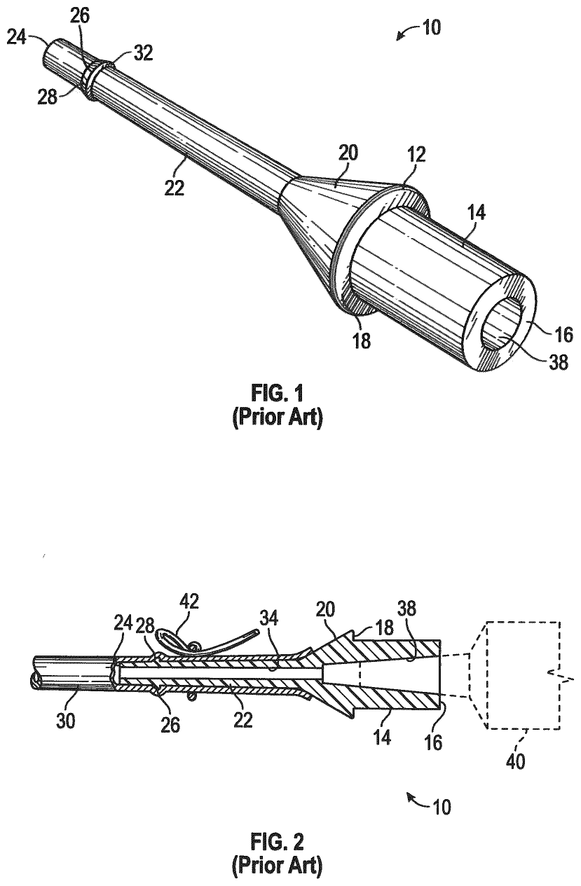

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Although multiple embodiments are shown and discussed in great detail, it will be apparent to those skilled in the relevant art that some features that are not relevant to the present invention may not be shown for the sake of clarity.

As used in this disclosure, the term “proximal” defines the end of the described embodiments opposite the cannula; that is, the axial direction opposite that of the cannula. The term “distal” similarly defines the cannula end of the described embodiments; that is, the axial direction towards the cannula.

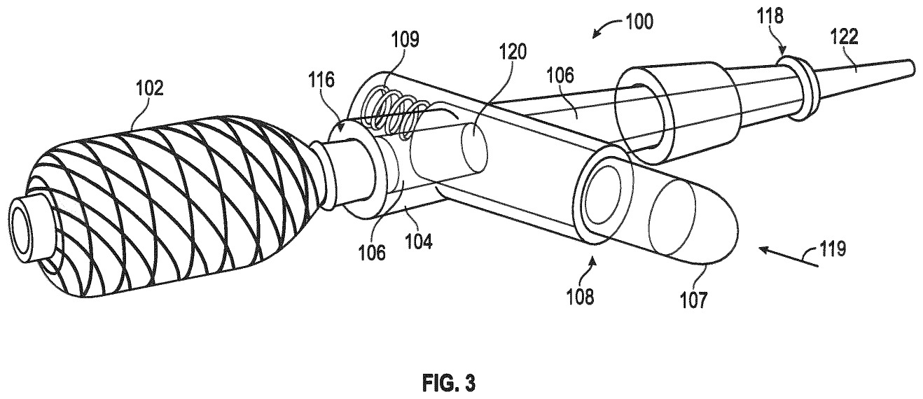

Referring to FIG. 3, the exemplary elastomeric vein graft preparation pump 100 (or “pump 100”) can be designed and constructed in accordance with the disclosed principles. As mentioned above, the pump 100 can provide regulated pressurization of the fluid pushed through harvested veins. This known (or predictable) pressurization can be set at a predetermined amount that is safe for the harvested vein section and advantageously eliminates the human error that too often causes over pressurization during distension of grafted veins due to manual pressurization.

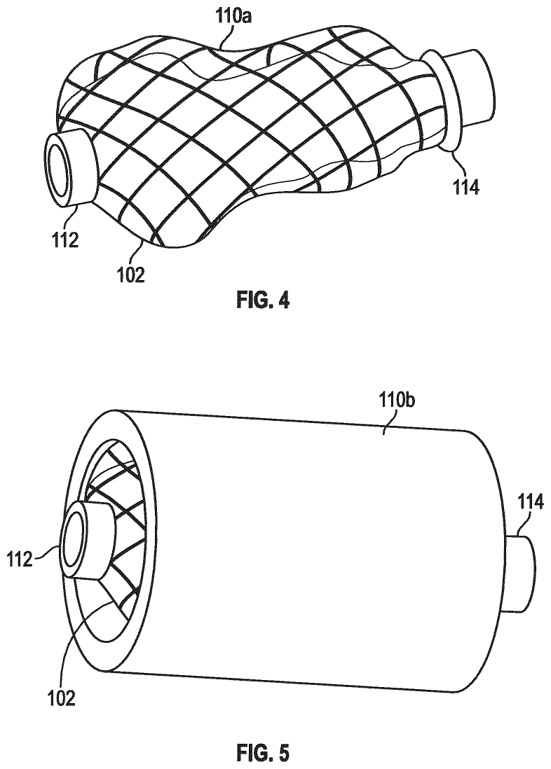

The components of the illustrated embodiment of the pump 100 can include an elastomeric compliant body 102. The elastomeric compliant body 102 can be sized and configured to securely hold a predetermined amount of fluid and provide a known (or predictable) pressure delivery profile, e.g., an initial pressure that decreases over time as the consistent fluid pressure for vein irrigation. The elastomeric compliant body 102 is described in more detail in FIG. 4 that follows. The pump 100 can also include a housing 104 that can define a fluid conduit 106 that connects a vein at a distal end of the pump 100 to the elastomeric compliant body 102. Thus, the fluid conduit 106 is configured to allow fluid to flow from the elastomeric compliant body 102 to the cannula 122 and into a vein. The fluid conduit 106 can be formed into a unitary component with housing 104. Alternatively, fluid conduit 106 and housing 104 can be separate components.

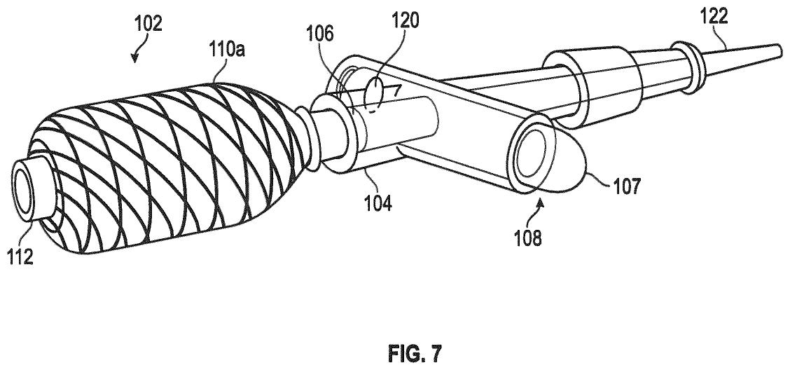

The pump 100 also includes a control valve 108 configured to control the flow of fluid through the fluid conduit 106. In one embodiment, control valve 108 can enable a user to manually control (e.g., start and stop) the flow of the fluid. The control valve 108 is disposed within the housing 104, which intersects the fluid conduit 106 such that a manual change in orientation of the control valve 108 prevents or allows the movement of fluid through the fluid conduit 106. In one embodiment, control valve 108 can be housed in a valve plug body (not illustrated) of housing 104. The control valve 108 can be a spring-loaded plug valve having a cylindrical (or tapered) plug 107 with an aperture 120 that passes through the plug 107. The aperture 120 can be configured to be slidably aligned with the fluid conduit 106 to allow flow or repositioned to block flow. In the open position, the control valve 108 is positioned such that the aperture 120 is aligned with the fluid conduit 106, allowing fluid to flow through the fluid conduit 106, from the elastomeric compliant body 102 toward the distal interface connector 118 that engages with the cannula 122. In the closed position, depicted in FIG. 7, the plug 107 is positioned such that the aperture 120 is not aligned with the fluid conduit 106. With the control valve 108 in the closed position, the fluid conduit 106 is impeded such that fluid may not flow through the fluid conduit 106, from the elastomeric compliant body 102 toward the distal interface connector 118. The control valve 108 can also be positioned in a partially open position, where a portion of the aperture 120 is aligned with fluid conduit 106. In such positions, the user can adjust the flow rate by increasing or decreasing the portion of the aperture 120 that is aligned with the fluid conduit 120.

In the depicted embodiment, the control valve 108 includes a spring 109 to bias the control valve 108 in the open position. A force applied to the end of the plug 107 in the direction of arrow 119 can overcome the spring force to cause the control valve 108 to assume the closed position. A locking mechanism (not shown) can maintain the control valve 108 in the closed position until a subsequent force, applied to the control valve 108 in the direction of arrow 119 can disengage the locking mechanism and allow the spring force to cause the control valve 108 to assume the open position. Plug 107 can include a plug retainer (not shown), which prevents the plug 107 from being ejected from the housing 104 by the spring 109.

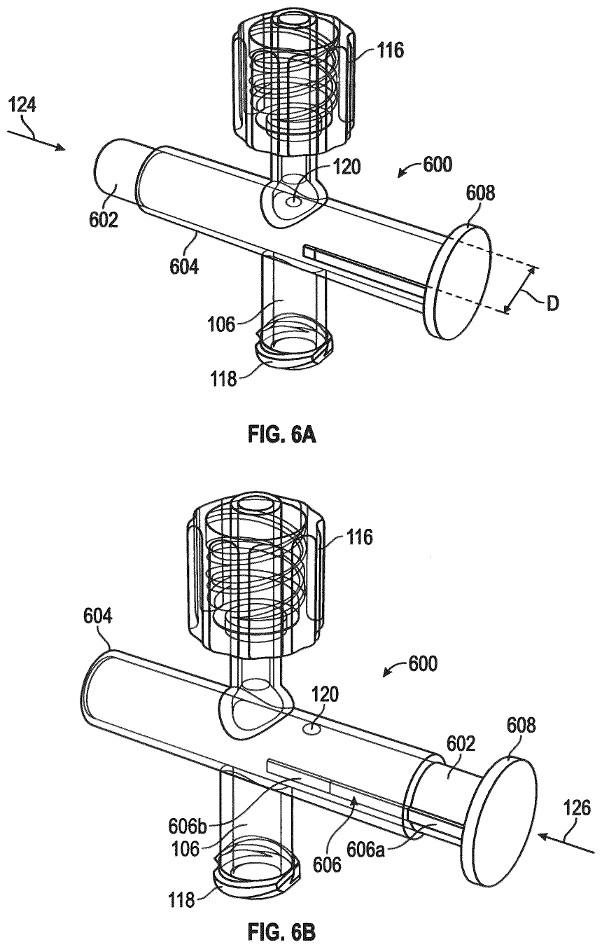

Many other embodiments of a control valve that can achieve the same utility are within the scope of the claims. Examples include but are not limited to ball valves, gate valves, butterfly valves, and globe valves. Another exemplary control valve 108 is shown and described in more detail in FIG. 6.

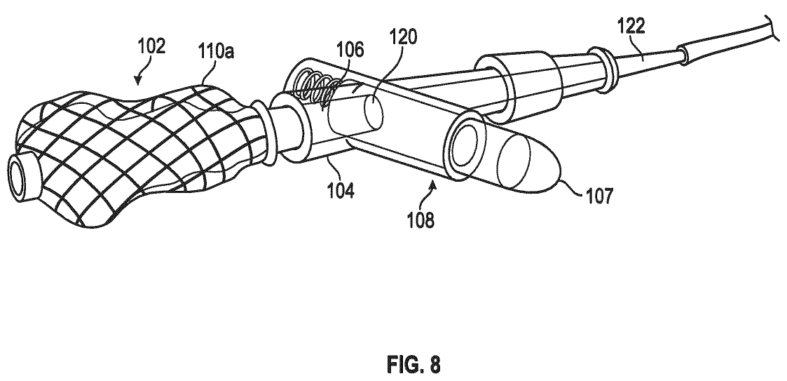

The pump 100 can also include an overfill prevention device 110 configured to prevent overfilling of the elastomeric compliant body 102 with fluid. A first example of the overfill prevention device 110a is shown and described in FIG. 4 and a second example of the overfill prevention device 110b is shown and described in FIG. 5. Overfill prevention devices may be generally referred to with reference numeral 110. Each of these components are discussed in greater detail below. Additionally, the functionality of the pump 100 as disclosed herein is described in detail with reference to FIGS. 7 and 8 below.

Referring to FIG. 4, an exemplary embodiment of an elastomeric compliant body 102 for use with the pump 100 is disclosed to illustrate one non-limiting embodiment. The illustrated embodiment of the elastomeric compliant body 102 includes a filling port 112 at the proximal end and a body connector 114 at the distal end. The body connector 114 can be sized and shaped to engage a connector interface 116 at the proximal end of the housing 104. The exemplary filling port 112 disclosed herein allows fluid to be received into the elastomeric compliant body 102 but prevents subsequent discharge of fluid from the filling port 112. In a first embodiment, the filling port 112 can be a needleless connector. Another embodiment of a filling port 112 could be comprised of a Luer taper slip style connector with a spring-loaded one-way check valve and a pressure-limiting relief valve. Many other embodiments of a filling port that can achieve the same utility are within the scope of the claims but are not presented here for the sake of brevity.

The elastomeric compliant body 102 can be formed from elastomers typically used in medical devices (e.g., rubber, silicone, polyurethane, nylon, latex) or other forms of elastomers that have desirable characteristics. The filling of the elastomeric compliant body 102 with a fluid causes a buildup of pressure in the elastomeric compliant body 102 and tension on the surface of the elastomeric compliant body 102 that can be used to force the liquid contained therein through the elastomeric compliant body connector 114 engaged with the proximal connector interface 116 of housing 104. To prevent overfilling of the elastomeric compliant body 102 with fluid, which could compromise the integrity of the elastomeric compliant body 102 or cause the elastomeric compliant body 102 to provide a pressure that exceeds a preferred pressure for distension of vein grafts, the expansion of the elastomeric compliant body 102 can be controlled by an overfill prevention device 110. One embodiment of an overfill prevention device 110a is a net or mesh surrounding the elastomeric compliant body 102 which can limit expansion of the elastomeric compliant body 102 to a certain predetermined size. The overfill prevention device 110a can have a collapsed state when the elastomeric compliant body 102 is empty, a partially expanded state when the elastomeric compliant body 102 is partially filled, and a fully expanded state when the elastomeric compliant body 102 is filled to the predetermined maximum volume.

Referring to FIG. 5, another exemplary embodiment of an overfill prevention device 110 is described. The overfill prevention device 110b can be a rigid body that surrounds (or constrains) the expansion of elastomeric compliant body 102. The overfill prevention device 110b can at least partially enclose the elastomeric compliant body 102 or fully enclose the elastomeric compliant body 102 while leaving the filling port 112 and the elastomeric compliant body connector 114 exposed. In the non-limiting embodiment depicted in FIG. 5, the overfill prevention device 110b is a cylindrical body opened at either end. The pump 100 can be configured to use one or both of the overfill prevention devices (110a, 100b).

FIGS. 6A and 6B illustrate an exemplary embodiment of a control valve 600 for use with the pump 100. In the depicted embodiment, the control valve 600 includes a plug 602 with a plug aperture 120 that passes through the plug 602 from one side to the other. The plug 602 is slidably engaged within a valve plug body 604 so that the control valve 600 can be placed in the open position, as shown in FIG. 6A, or in the closed position, as shown in FIG. 6B. In an embodiment in which the plug 602 is cylindrical and the valve plug body 604 defines a cylindrical cavity sized to receive the plug 602, control valve 600 can include an alignment interface 606 to prevent the plug 602 from rotating within the valve plug body 604. In the illustrative embodiments, the alignment interface 606 can include an alignment rail 606a that is received within an alignment channel 606b.

Extending outwardly from the valve plug body 604 is a pair of connection interfaces 116 and 118. By attaching elastomeric compliant body 102 to the proximal connector interface 116 and the cannula 122 to distal connector interface 118, pump 100 can be formed. Although connection interfaces 116 and 118 are depicted as screw-type interfaces, the examples shown in FIGS. 6A and 6B are non-limiting. Thus, the connection interfaces 116 and 118 can be any currently existing or later developed form of connector.

The plug 602 can include a plug head 608, which can receive a pressing force in the direction of arrow 126, shown in FIG. 6B, to cause the control valve 600 to transition from the closed configuration to the open configuration. In the depicted embodiment, the plug head 608 has a diameter D that is larger than a diameter of the valve plug body 604 to limit travel of the plug 602 so that fully depressing the plug head 608 causes the aperture 120 to align with the fluid conduit 106.

The control valve 600 is in the open position when the control valve aperture 120 is aligned with the fluid conduit 106. In the depicted embodiment, the control valve 600 is in the open position when the plug 602 is maximally depressed. The control valve 600 will remain in the open position until a force applied to the plug 602 in the direction of arrow 124, shown in FIG. 6A, causes the control valve 600 to assume the closed position, i.e., when the control valve aperture 120 is not aligned with the fluid conduit 106. Alternatively, a grasping force applied to the plug head 608 and exerted in the general direction of arrow 124 can also cause the control valve 600 to assume the closed position. In the closed position, the aperture 120 is not aligned with the fluid conduit 106, which prevents fluid flow past plug 602. The control valve 600 can also be positioned in a partially open position, where a portion of the aperture 120 is aligned with fluid conduit 106. In such positions, the user can adjust the flow rate by increasing or decreasing the portion of the aperture 120 that is aligned with the fluid conduit 120.

Referring to FIG. 7, a perspective view of the pump 100 in a loaded, unused state is illustrated. In the illustrated loaded, unused state, the elastomeric compliant body 102 is shown filled with fluid. In this position, the elastomeric compliant body 102 is extended, at maximum to the extent permitted by the overfill prevention device 110a. Additionally, the control valve 108 is positioned in the closed position, where the fluid conduit 106 is impeded such that fluid may not flow past the control valve 108. To load the pump 100, the user may manually fill the elastomeric compliant body 102 by affixing a syringe or other source of fluid to the filling port 112 and supplying fluid to the elastomeric compliant body 102. As a result, forces are created, e.g., fluid pressure and surface tension, which can be used to expel the fluid from the elastomeric compliant body 102.

Referring to FIG. 8, a perspective view of the pump 100 in a deployed, used state is illustrated. The pump 100 can be transitioned from the unused state, shown in FIG. 7, to the used state by operating the control valve 108 and moving it from a closed position to the open position. In the open position, fluid is permitted to flow through the fluid conduit 106 from the elastomeric compliant body 102 to the cannula 122 at a controlled pressure.

In accordance with the disclosed principles, the distended elastomeric compliant body 102 allows for a controlled release of fluid in a known (or predictable) manner. Pressure is limited via use of the elastomeric compliant body 102 and the overfill prevention device 110. For the elastomeric compliant body 102, the elastic force of the elastomeric compliant body 102 can be selected to provide the desired or required pressure, with a stiffer elastomeric compliant body 102 contributing to a greater amount of pressure, while a less stiff elastomeric compliant body 102 contributes to a lower amount of pressure of the fluid injected into the harvested vein for irrigation. For the overfill prevention device 110, the maximum amount of fluid allowed to fill the elastomeric compliant body 102 can be selected to provide the desired or required pressure, with a greater fluid allowance providing a greater amount of pressure, while a lesser fluid allowance will provide a lower pressure of the fluid injected into the harvested vein for irrigation. The provision of known (or predictable) pressurization at a predetermined amount safe for the harvested vein section advantageously eliminates the human error present during distension of grant veins with manual pressurization. In some embodiments, the viscosity of the fluid used in preparing a vein graft can be considered in determining the desired amount of pressure provided by the elastomeric compliant body 102 and allowed by the overfill protection device 110.

Also, as described above, the pump 100 as disclosed herein can include an on/off mechanism (e.g., the control valve 108) that allows delivery of fluids into the harvested vein to be stopped and started at any time. This stopping capability allows for initial filling of the pump 100 as well as the ability to reduce or eliminate internal pressure to the vein if perforations in the vein are detected during irrigation/preparation. As such, any such perforations may be stitched or otherwise repaired while the pressure from the pump 100 is eliminated by the on/off mechanism.

While this disclosure has been particularly shown and described with reference to preferred embodiments, it will be understood by those skilled in the pertinent field art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. An elastomeric vein graft preparation pump, comprising:
an elastomeric compliant body configured to contain a fluid;
a fluid conduit coupled to the elastomeric compliant body; and
a control valve disposed through the fluid conduit, the control valve configured to regulate a flow of the fluid through the fluid conduit, wherein:
the elastomeric compliant body provides an elastic recoil pressure based on a volume of the fluid contained in the elastomeric compliant body, and
the fluid flows through the fluid conduit at a pressure based on the elastic recoil pressure.

2. The elastomeric vein graft preparation pump of claim 1, further comprising:
an overfill prevention device configured to limit expansion of the elastomeric complaint body.

3. The elastomeric vein graft preparation pump of claim 2, wherein the overfill prevention device is flexible body that at least partially surrounds the elastomeric compliant body.

4. The elastomeric vein graft preparation pump of claim 3, wherein the overfill prevention device is a mesh or net.

5. The elastomeric vein graft preparation pump of claim 2, wherein the overfill prevention device is a rigid body that at least partially surrounds the elastomeric compliant body.

6. The elastomeric vein graft preparation pump of claim 5, wherein the overfill prevention device is a cylindrical housing sized to allow each end of the elastomeric compliant body to be exposed.

7. The elastomeric vein graft preparation pump of claim 1, further comprising:
a housing body defining the fluid conduit, wherein the housing body includes a first connector configured to connect to the elastomeric compliant body and a second connector configured to connect to a vessel cannula.

8. The elastomeric vein graft preparation pump of claim 7, wherein the elastomeric compliant body comprises at least one of a filling port configured to be connected to a source of the fluid, and a body connector configured to connect the elastomeric compliant body to the housing body.

9. The elastomeric vein graft preparation pump of claim 8, wherein the filling port is one of a needless connector or a Luer taper.

10. The elastomeric vein graft preparation pump of claim 1, wherein:
the control valve comprises a spring and a plug,
the plug comprises an aperture formed through the plug, and
the plug is biased by the spring to achieve one of a closed position with the aperture misaligned with the fluid conduit or an open position with the aperture aligned with the fluid conduit.

11. The elastomeric vein graft preparation pump of claim 7, wherein:
the control valve comprises a guide rail, and
the guide rail maintains an orientation of the plug.

12. A method of operating an elastomeric vein graft preparation pump, the method comprising:
at least partially filling an elastomeric compliant body with a fluid;
coupling the elastomeric compliant body to a first end of a housing that defines a fluid conduit extending through the housing from the first end to a second end, wherein:
the housing includes a control valve disposed through the fluid conduit, and
the control valve is configured to selectively release the fluid from the elastomeric compliant body;
coupling a vessel cannula to the second end of the housing; and
coupling a first end of a vessel to the vessel cannula, wherein the vessel is sealed at a second end.

13. The method of claim 12, wherein coupling the first end of the vessel to the vessel cannula further comprises sealing the second end of the vessel.

14. The method of claim 13, wherein filling the elastomeric compliant body with the fluid comprises:
attaching a source of the fluid to a filling port of the elastomeric compliant body; and
introducing the fluid into the elastomeric compliant body.

15. The method of claim 14, wherein:
the source of the fluid is a syringe and the filling port is a Luer taper or a needleless connector,
attaching the syringe with the elastomeric compliant body includes coupling the syringe with the Luer taper or the needleless connector, and
introducing the fluid comprises depressing a plunger of the syringe.

16. The method of claim 12, wherein at least partially filling the elastomeric compliant body comprises filling the elastomeric complaint body with a predetermined volume of the fluid.

17. The method of claim 16, wherein the predetermined volume of the fluid is determined by an overfill prevention device engaged at least partially around the elastomeric compliant body.

18. The method of claim 17, wherein the overfill prevention device is a flexible body or a rigid body.

19. The method of claim 12, further comprising:
operating the control valve to release at least some of the fluid from the elastomeric compliant body at a pressure based on a volume of the fluid in the elastomeric compliant body.

20. The method of claim 19, wherein:
the control valve comprises a spring and a plug,
the plug comprises an aperture formed through the plug,
the spring provides a biasing force to the plug to achieve one of a closed position with the aperture misaligned with the fluid conduit or an open position with the aperture aligned with the fluid conduit, and
operating the control valve comprises providing a compressive force to the plug to overcome the biasing force of a spring to selectively release the fluid from the elastomeric compliant body.
